(19) United States
(12) Patent Application Publication
(10) Pub. No.: US 2022/0062595 A1
(43) Pub. Date: Mar. 3, 2022

(54) MEDICAL GUIDEWIRE

(71) Applicant: Hua SHANG, Nanjing (CN)

(72) Inventor: Hua SHANG, Nanjing (CN)

(21) Appl. No.: 17/406,914

(22) Filed: Aug. 19, 2021

Related U.S. Application Data
(63) Continuation of application No. PCT/CN2020/134611, filed on Dec. 8, 2020.

(30) Foreign Application Priority Data
Aug. 31, 2020 (CN) CN202010894225.1

(51) Int. Cl. A61M 25/09

(52) U.S. Cl. CPC A61M 25/09041; A61M 2025/09116; A61M 2025/09133

(57) ABSTRACT

The disclosure provides a medical guidewire. The medical guidewire includes an equal-diameter fiber and a variable-diameter sleeve surrounding the equal-diameter fiber. The variable-diameter sleeve includes a shaping section, a supporting section, and a pushing section that are connected in sequence. The shaping section, the supporting section and the pushing section have outer diameters increased sequentially. An asymmetric structure is provided on the variable-diameter sleeve itself or the surround of the variable-diameter sleeve along the equal-diameter fiber. The medical guidewire provided by the disclosure has improved bending performance and operability, so that it is easy to manipulate the medical guidewire to enter blood vessels with a large opening angle. thereby improving the effect of minimally invasive interventional treatment.

### CROSS-REFERENCE TO RELATED APPLICATIONS

This patent application is a continuation of PCT/CN2020/134611 filed Dec. 8, 2020, which claims priority to Chinese Application CN202010894225.1 filed Aug. 31, 2020, which applications are incorporated herein by specific reference in their entirety.

### TECHNICAL FIELD

The disclosure relates to the technical field of medical devices, in particular to a medical guidewire.

### BACKGROUND

Minimally invasive interventional therapy is a medical technology that uses specific devices such as a puncture needle, guidewire or catheter under imaging guidance to accurately reach lesion sites for diagnosis and treatment without open surgery to human body. Minimally invasive interventional therapy is more and more favored by patients for its characteristic such as definite curative effect, fast recovery, strong targeting, low recurrence, no side effects, small trauma, safety and reliability, low cost and the like.

The medical guidewire is frequently used in clinical practice. For example, the guidewire may be used for assisting the installation of heart stents, the ablation of thrombus, and the treatment of tumor embolization. As for the interventional surgery, the safety of the guidewire comes first. Therefore, the guidewire must have many characteristics, such as flexible head portion, good compliance, no damage, high plasticity, and providing low to moderate support.

At present, the medical guidewire sold on the market is usually composed of a wire made of stainless steel having multiple diameters in a core. Such wire of stainless steel is formed to the medical guidewire by winding from a top end. However, this solution will cause the guidewire to have a larger diameter, making it difficult to enter blood vessel having a small diameter.

Therefore, how to improve the operability of the medical guidewire while ensuring that the medical guidewire can enter blood vessels having smaller diameter and branch blood vessels having a larger opening angle has become an urgent problem to be solved.

### SUMMARY

In view of the above, embodiments of the disclosure provide a medical guidewire, so as to solve technical defects in the related art.

The disclosure provides a medical guidewire. The medical guidewire includes an equal-diameter fiber and a variable-diameter sleeve surrounding the equal-diameter fiber. The variable-diameter sleeve includes a shaping section capable of bending, a supporting section capable of supporting an advancement of the shaping section, and a pushing section connected to an operating handle. The shaping section, the supporting section and the pushing section are connected in sequence and outer diameters of the shaping section, the supporting section and the pushing section are increased sequentially. The medical guidewire is further provided with an asymmetric structure capable of directionally bending the medical guidewire to one side.

Optionally, the equal-diameter fiber is located at an axis of the variable-diameter sleeve, and the asymmetric structure is an asymmetric tube wall structure of the variable-diameter sleeve.

Optionally, the asymmetric tube wall structure is an asymmetric slit opened on the shaping section of the variable-diameter sleeve. The asymmetric slit is a spiral slit, and has a width that gradually decreases with spiral patterns along a direction from the shaping section to the pushing section; or is a rectangular slit, and has a depth on one side of the variable-diameter sleeve less than a depth on other side of the variable-diameter sleeve.

Optionally, the asymmetric structure of the variable-diameter sleeve lies in an inner wall of the variable-diameter sleeve. An asymmetric array gap structure capable of directionally bending the medical guidewire to one side is formed between the inner wall of the variable-diameter sleeve and the equal-diameter fiber.

Optionally, the asymmetric tube wall structure is formed by a wall thickness of one side of the variable-diameter sleeve being smaller than a wall thickness of other side of the variable-diameter sleeve.

Optionally, the asymmetric tube wall structure is formed by a shape of the variable-diameter sleeve having a convex side and a planar side, or having a convex side and a concave side. The convex side has an arched structure.

Optionally, the equal-diameter fiber is fixed on the inner wall on one side of the variable-diameter sleeve.

Optionally, the medical guidewire further includes a transition section with a gradually changing diameter. The transition section is located between the shaping section and the supporting section, and a diameter of the transition section gradually increases along a direction from the shaping section to the supporting section.

Optionally, a proximal end of the medical guidewire is an operating handle, and the operating handle is provided with a tensing device for applying pulling force on the equal-diameter fiber. A distal end of the medical guidewire has a hemispherical structure. The proximal end of the medical guidewire is connected to the pushing section of the variable-diameter sleeve, and the distal end of the medical guidewire is connected to the equal-diameter fiber and the shaping section of the variable-diameter sleeve.

Optionally, a polymer layer is provided outside the variable-diameter sleeve, and is a hydrophilic coating or a hydrophobic coating.

Optionally, the variable-diameter sleeve is a variable-diameter hypotube, has an outer diameter of 0.6-1.0 mm, and has an inner diameter of 0.1-0.5 mm.

The medical guidewire provided in this disclosure includes an equal-diameter fiber and a variable-diameter sleeve surrounding the equal-diameter fiber. The variable-diameter sleeve includes a shaping section, the supporting section and the pushing section which are connected in sequence. The outer diameters of the shaping section, the supporting section and the pushing section are increased sequentially. Among them, the shaping section has the smallest diameter, and thus is more bendable compared to the supporting section and the pushing section, which prompts the shaping section to guide the whole guidewire to advance along the bent blood vessels. The diameter of the supporting section is larger than the diameter of the shaping section, which can make it have sufficient elasticity to support the movement of the shaping section in blood vessels. The diameter of the pushing section is larger than the diameters of the shaping section and the supporting section, which can make it have sufficient rigidity to provide forward driving force to the shaping section and the supporting section in blood vessels. In addition, in order to improve the bending performance and operability of the medical guidewire, the asymmetric structure is provided on the variable-diameter sleeve itself or the surround of the variable-diameter sleeve along the equal-diameter fiber, so that it is easy to manipulate the medical guidewire to enter smaller blood vessels and blood vessels with the large opening angle, thereby improving the effect of minimally invasive interventional treatment.

### BRIEF DESCRIPTION OF THE DRAWINGS

FIG. 1 is a schematic diagram of the structure of the medical guidewire according to an example of the disclosure;

FIG. 2 is a schematic diagram of the structure of the medical guidewire according to an example of the disclosure;

FIG. 3 is a schematic diagram of the structure of the medical guidewire according to an example of the disclosure;

FIG. 4 is a schematic diagram of the structure of the medical guidewire according to an example of the disclosure;

FIG. 5 is a local schematic diagram of the medical guidewire according to an example of the disclosure;

FIG. 6 is a cross-sectional schematic diagram of the medica guidewire according to an example of the disclosure;

FIG. 7 is a local schematic diagram of the medical guidewire according to an example of the disclosure.

### LIST OF REFERENCE SYMBOLS

1, variable-diameter sleeve; 2, equal-diameter fiber; 3, shaping section; 4, transition section; 5, supporting section; 6, pushing section; 7, hemispherical structure; 8, polymer layer; 9, array gap structure; 10, tube wall; 11, convex side; 12, planar side; 13, grating assembly; 14, core layer; 15, cladding layer.

### DETAILED DESCRIPTION

The embodiments of the disclosure are described below with reference to the drawings.

In this disclosure, unless otherwise specified, the scientific and technical terms used herein have the meanings commonly understood by those skilled in the art. In addition, the reagents, materials and operation steps used in this disclosure belong to common reagents, materials and routine procedures widely used in the corresponding field. Furthermore, in order to better understand the disclosure, definitions and explanations of related terms are provided below.

In this disclosure, hypotube refers to a long metal tube with micro-engineering characteristics throughout the tube. It is an important component of a catheter for minimally invasive treatment and is used to dredge clogged arteries in conjunction with a balloon and a stent. The balloon of the catheter is attached to a distal end of the hypotube. The hypotube enters the human body and pushes the balloon toward the clog portion in the artery along the long tortuous blood vessel. During this operation, it is required to avoid the kinking of the hypotube, and enable it to move smoothly in the human body (propulsion, tracking and rotation).

In this disclosure, Seldinger puncture technique refers to a percutaneous vascular puncture process, and is mainly used in catheterization. This technique includes the following main operations: performing the venipuncture by using 21 Ga injection needle; after entering the blood vessel, inserting a guidewire from the needle hole to a junction of the superior vena cava and the right atrium under fluoroscopy; removing the needle, and expanding the puncture point by using a scalpel; pushing the vasodilator along the guidewire and removing it after expanding the skin; inserting a catheter along the guidewire to the junction of the superior vena cava and the right atrium.

In this disclosure, the modified Seldinger puncture technique includes the following main operations: performing the venipuncture by using a trocar or small needle; inserting guidewire through the cannula and puncture needle; pulling out the cannula or puncture needle; cutting the skin by a scalpel, and expanding the puncture site; inserting a incubator (dilator/intubation sheath) (tearable type) along the guidewire; pulling out the guidewire and dilator, leaving the intubation sheath; and inserting a catheter by the cannula sheath to a predetermined length.

### Example 1

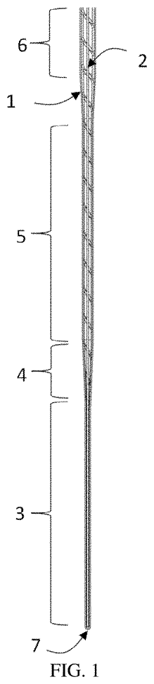

The present example provides a medical guidewire. As shown in FIG. 1, the medical guidewire includes an equal-diameter fiber 2 and a variable-diameter sleeve 1 surrounding the equal-diameter fiber 2. The variable-diameter sleeve 1 includes a shaping section 3 capable of bending, a supporting section 5 capable of supporting advancement of the shaping section 3, and a pushing section 6 connected to an operating handle and used for driving the movement of the medical guidewire. The shaping section 3, the supporting section 5 and the pushing section 6 are connected in sequence and the outer diameters thereof are increased sequentially. The medical guidewire is further provided with an asymmetric structure capable of directionally bending the medical guidewire to one side.

The equal-diameter fiber 2 is a fiber with the same diameter throughout its length, may be located at an axis of the medical guidewire or fixed on one side of the variable-diameter sleeve. The variable-diameter sleeve 1 has a tubular structure with a gradually changing diameter, and sheathed outside the equal-diameter fiber 2. The shaping section 3, supporting section 5 and pushing section 6 of the variable-diameter sleeve 1 may all be equal-diameter sections or variable-diameter sections, depending on specific circumstances, which is not limited in this disclosure. In the case that the shaping section 3, supporting section 5 or pushing section 6 is the variable-diameter section, each section has the diameter gradually increased along the direction from the shaping section 3 to the supporting section 5. However, regardless of whether the shaping section 3, the supporting section 5 and pushing section 6 are equal-diameter sections or variable-diameter sections, the outer diameters thereof are different. The shaping section 3 has the smallest outer diameter, and the pushing section 6 has the largest outer diameter.

On this basis, the medical guidewire provided in this example may further includes a transition section 4 located between the shaping section 3 and the supporting section 5. A diameter of the transition section 4 gradually increases along the direction from the shaping section 3 to the supporting section 5.

In this example, the asymmetric structure of the medical guidewire is capable of directionally bending the medical guidewire to one side. The asymmetric structure is an asymmetric tube wall structure of the variable-diameter sleeve 1, such as an asymmetric slit, asymmetric array gap structure, asymmetric tube wall thickness or shape, etc. on the tube wall of the variable-diameter sleeve 1.

Specifically, a proximal end of the medical guidewire is an operating handle, and the operating handle is provided with a tensing device for applying pulling force on the equal-diameter fiber. A distal end of the medical guidewire has a hemispherical structure 7. The proximal end of the medical guidewire is connected to the pushing section 6 of the variable-diameter sleeve 1, and the distal end of the medical guidewire is connected to the equal-diameter fiber 2 and the shaping section 3 of the variable-diameter sleeve 1.

In the practices, the medical guidewire preferably has a total length of 2 m. The operating handle is preferably a prismatic structure, which is easy for rotating and pushing. The variable-diameter sleeve 1 has an outer diameter of 0.6-1.0 mm, and an inner diameter of 0.1-0.5 mm; the pushing section 6 has an outer diameter of preferably 0.8 mm, an inner diameter of preferably 0.4 mm, and a length of preferably 1 m. The supporting section 5 has an outer diameter of preferably 0.4 mm, an inner diameter of preferably 0.3 mm, and a length of preferably 0.8 m. The transition section 4 has a length of preferably 0.1 m. The shaping section 3 has an outer diameter of preferably 0.2 mm, an inner diameter of preferably 0.15 mm, and a length of preferably 0.1 m. The variable-diameter sleeve 1 is preferably a variable-diameter hypotube that is preferably made of Medical 304 stainless steel. In this example, the diameter of the medical guidewire reaches the millimeter level, so that the guidewire can safely enter the smaller blood vessels for detection or treatment, avoid the damage of the guidewire to walls of blood vessels, and has a wide range of applications.

The equal-diameter fiber 2 can be made of metal materials such as stainless steel, or an alloy composed of multiple metals, such as nickel-titanium alloy, aluminum alloy, white alloy, or non-metallic materials selected from plastic fiber, quartz fiber, carbon fiber and the like, preferably Medical 304 stainless steel. The equal-diameter fiber 2 has a length of 2 m, and a diameter of preferably 0.1 mm. The equal-diameter fiber 2 has a polished surface. One end of the equal-diameter fiber 2 is connected to the operating handle and the other end is connected to the hemispherical structure 7 at the distal end of the medical guidewire. A tensing device is fixed on the operating handle for applying driving force on the equal-diameter fiber 2 to make it tensioned.

The hemispherical structure 7 at the distal end of the medical guidewire can be made of metal materials or an alloy composed of multiple metals, such as nickel-titanium alloy, aluminum alloy, white alloy, or non-metallic materials selected from plastic, quartz, gem crystal, polymer and the like, which is not limited in the present disclosure. Preferably, a stainless steel may be used. The hemispherical structure 7 is connected to the distal end of the shaping section 3 of the variable-diameter sleeve 1 and the equal-diameter fiber 2 by welding.

In addition, a polymer layer 8 is provided outside the variable-diameter sleeve 1, and may be a hydrophilic coating or a hydrophobic coating. The hydrophilic coating can capture water to form a “gel-like” surface on the surface of the guidewire, reducing a resistance during the passage of the guidewire. The hydrophobic coating can resist water molecules to form a “waxy” surface, reducing friction during the passage, and enhancing the tracking of the guidewire.

In the practices, the tensing device on the operating handle can be appropriately loosened to keep the shaping section 3 straight, and the medical guidewire of the present example can be introduced into blood vessel by the Sedinger puncture or the modified Sedinger puncture technique, and is pushed along blood vessel through the operating handle. When it is necessary to pass through branch blood vessels, especially branch blood vessels with a large bending angle, the shaping section 3 is bent by tightening the tensing device, and the guidewire is rotated by rotating the operating handle, so that the shaping section 3 in bent state enters the branch blood vessels, and other portions of the guidewire is led into the branch blood vessels.

The medical guidewire provided in this example includes the equal-diameter fiber 2 and the variable-diameter sleeve 1 surrounding the equal-diameter fiber 2. The variable-diameter sleeve 1 includes the shaping section 3, the supporting section 5 and the pushing section 6 which are connected in sequence. The outer diameters of the shaping section 3, the supporting section 5 and the pushing section 6 are increased sequentially. Among them, the shaping section 3 has the smallest diameter, and thus is more bendable compared to the supporting section 5 and the pushing section 6, which prompts the shaping section 3 to guide the whole guidewire to advance along the bent blood vessel. The diameter of the supporting section 5 is larger than the diameter of the shaping section 3, which can make the supporting section 5 have sufficient elasticity to support the movement of the shaping section 3 in blood vessels. The diameter of the pushing section 6 is larger than the diameters of the shaping section 3 and the supporting section 5, which can make it have sufficient rigidity to provide forward driving force for the shaping section 3 and the supporting section 5 in blood vessels. In addition, in order to improve the bending performance and operability of the medical guidewire, the asymmetric structure is provided on the variable-diameter sleeve 1 itself or the surround of the variable-diameter sleeve 1 along the equal-diameter fiber 2, so that it is easy to manipulate the medical guidewire to enter blood vessels with the large opening angle.

### Example 2

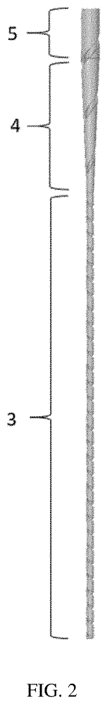

On the basis of example 1, the present example provides a medical guidewire provided with the asymmetric structure capable of directionally bending the medical guidewire to one side. as shown in FIG. 2. The asymmetric structure is an asymmetric tube wall structure of the variable-diameter sleeve 1. The asymmetric tube wall structure is an asymmetric slit opened on the variable-diameter sleeve 1. The asymmetric slit is a spiral slit, and has a width that gradually decreases with spiral patterns along the direction from the shaping section 3 to the pushing section 6.

Specifically, the spiral slit of the variable-diameter sleeve 1 can be formed through rotary cutting by laser cutting process. The spiral slit on other sections except to the shaping section 3 has a thread pitch of preferably 1 mm, and a width of preferably 0.5 mm. The spiral slit on the shaping section 3 changes continuously, and slit on one side is preferably 0.1 mm, and on the other side is of preferably 0.5 mm.

In the medical guidewire of this example, the spiral slit of the asymmetric structure of the variable-diameter sleeve 1 can make the shaping section 3 have asymmetric mechanical properties. Therefore, the medical guidewire will be bent toward one side when force is applied, and thus it can easily and quickly enter branch blood vessels having a large opening angle. The spiral slit has excellent global coherence, which can improve the flexibility of the variable-diameter sleeve 1 and the medical guidewire, reduce the abrasion produced during use and prolong service life thereof.

### Example 3

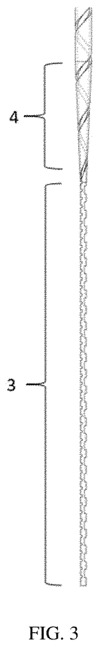

On the basis of example 1, the present example provides a medical guidewire provided with an asymmetric structure capable of directionally bending the medical guidewire to one side, as shown in FIG. 3. The asymmetric structure is an asymmetric tube wall structure of the variable-diameter sleeve 1. The asymmetric tube wall structure is an asymmetric slit opened on the variable-diameter sleeve 1. The asymmetric slit is a rectangular slit, and has a depth on one side of the variable-diameter sleeve 1 less than a depth on other side of the variable-diameter sleeve 1.

In the medical guidewire of this example, the rectangular slit of the asymmetric structure of the variable-diameter sleeve 1 can make the shaping section 3 have asymmetric mechanical properties. Therefore, the medical guidewire will be bent toward one side having a deeper depth when force is applied, and thus it can easily and quickly enter branch blood vessels having a large opening angle. In addition, the rectangular slit is simple in the manufacture, is easy to be controlled during the use, and has high maneuverability and wide applications.

In addition, the rectangular slit described in this example and the spiral slit described in example 2 can provided in combination on the same medical guidewire, which can further improve the flexibility of the medical guidewire in clinical application. For example, the rectangular slit is applied to the shaping section 3, and the spiral slit is applied to the transition section 4, depending on specific circumstances, which is not limited in this disclosure.

### Example 4

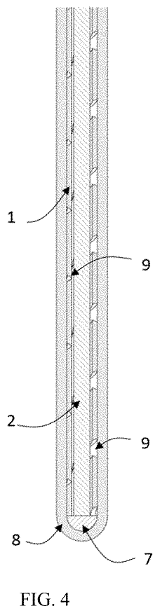

On the basis of example 1, the present example provides a medical guidewire provided with an asymmetric structure capable of directional bending of the medical guidewire, as shown in FIG. 4. The asymmetric structure is an asymmetric tube wall structure of the variable-diameter sleeve 1. The asymmetric tube wall structure is an asymmetric array gap structure 9 capable of directionally bending the medical guidewire to one side formed between the inner wall of the variable-diameter sleeve and the equal-diameter fiber 2.

Specifically, a plurality of asymmetric grooves arranged in an array may be formed on the inner wall of the variable-diameter sleeve 1 through cutting by means of laser cutting. These grooves can be in various shapes, such as square, ellipse, triangle, spiral, preferably in shape of spiral. The asymmetric array gap structure 9 may be formed between grooves on the inner wall of the variable-diameter sleeve 1 and the equal-diameter fiber 2.

In the medical guidewire of this example, the asymmetric array gap structure 9 of the variable-diameter sleeve 1 may effectively enhance the flexibility of the variable-diameter sleeve 1, thereby enhancing the flexibility of the medical guidewire. Therefore, the medical guidewire can be adjusted correspondingly according to the change of the blood vessel path during use, and the compliance of the medical guidewire in the blood vessel is improved, thereby improving the treatment effect thereof.

### Example 5

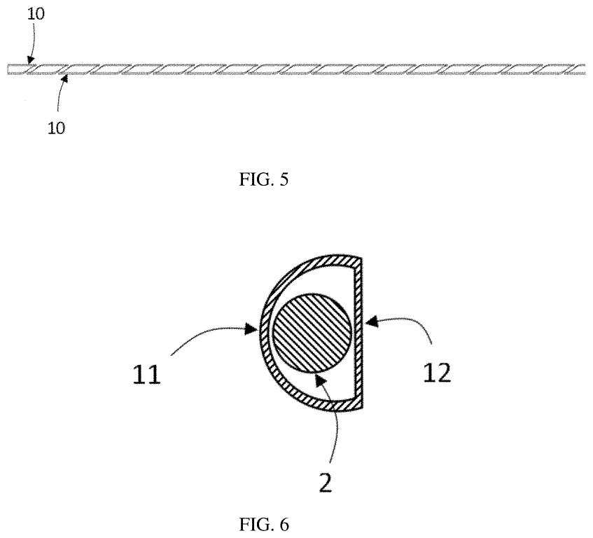

On the basis of example 1, the present example provides a medical guidewire provided with an asymmetric structure capable of directional bending of the medical guidewire, as shown in FIG. 5. The asymmetric tube wall structure of the variable-diameter sleeve 1 is formed by a wall thickness of the tube wall 10 of the variable-diameter sleeve 1. It is formed by a thickness of the tube wall 10 of one side of the variable-diameter sleeve 1 being smaller than a thickness of the tube wall 10 of the other side.

Specifically, taking the variable-diameter sleeve 1 being a cylindrical sleeve as an example, if it is divided into two half-cylindrical sleeves along the cross-sectional diameter, one half-cylindrical sleeve has the tube wall 10 having a thinner thickness which is preferably 0.1 mm-0.3 mm, while the other half-cylindrical sleeve has the tube wall 10 having a thicker thickness which is preferably 0.3 mm-0.5 mm.

In the medical guidewire provided in this example, the variable-diameter sleeve 1 has the tube wall 10 having the thinner thickness on one side, and has the tube wall 10 having the thicker thickness on the other side. When the medical guidewire is stressed, it will bend to the side of tube wall having the thinner thickness, so as to advance into the blood vessel with a larger opening angle.

### Example 6

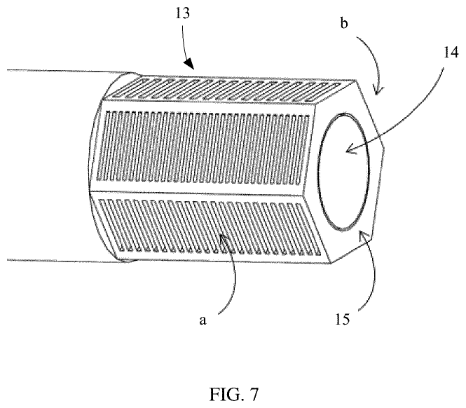

On the basis of example 1, the present example provides a medical guidewire provided with an asymmetric structure capable of directional bending of the medical guidewire, as shown in FIG. 6. The asymmetric tube wall structure of the variable-diameter sleeve 1 is formed by the shape of the variable-diameter sleeve 1. It is formed by a convex side 11 and a planar side 12 of the variable-diameter sleeve 1, or the convex side 11 and a concave side. The convex side 11 has an arched structure.

Specifically, because the convex side 11 has the arched structure and its rigidity is relatively strong, when the medical guidewire is stressed, it will bend to the concave side or the planar side1 12 opposite to the convex side 11, thereby making the medical guidewire advance into the branch blood vessel more smoothly.

It is noted that the asymmetric structure provided in examples 2-6 may be used in one medical guidewire or one section of the variable-diameter sleeve 1. For example, the spiral slit provided by example 2 and the asymmetric tube wall 10 thickness provided by example 5 may be used in combination in the shaping section 3; or the spiral slit provided by example 2, the asymmetric tube wall 10 thickness provided by example 5 and the asymmetric tubular structure provided by example 6 may be used in combination in the shaping section 3, depending on specific circumstances, which is not limited in this disclosure.

### Example 7

On the basis of example 1, the present example provides a medical guidewire as shown in FIG. 7. The equal-diameter fiber 2 may be an optical fiber, and, at end close to the shaping section 3, of equal-diameter fiber 2, may be provided with at least one grating assembly 13. The grating assemblies 13 are sleeved on the equal-diameter fiber 2 at intervals, and are arranged longitudinally along the equal-diameter fiber 2.

Specifically, the equal-diameter fiber 2 includes a core layer 12 located at the axis and a cladding layer 15 wrapped around the core layer 12. The grating assemblies 13 are sleeved outside the cladding layer 15 at intervals. Each of grating assemblies 13 is in shape of hollow prism. The grating assembly 13 includes a plurality of gratings with different periods, and each grating constitutes a side surface of the grating assembly. When pulsed lasers with multiple wavelengths are transmitted into the optical fiber, the wavelengths of the pulses coupled from different gratings are different. The number of gratings of the grating assembly is the same as the number of side surfaces of the prism. For example, when the grating assembly is in the shape of a hollow hexagonal prism, it is composed of 6 gratings with different periods. In the practices, there may be three grating assemblies, and each of the grating assemblies has six gratings.

The grating is an optical device specially for emitting and collecting laser light, and is composed of a large number of parallel slits having equal width and equal spacing. In the optical fiber guidewire described in this example, laser light transmitted by the optical fiber guidewire can be scattered into the body cavity through the grating assemblies 13, and retro-reflected laser light can also be collected through the grating assemblies 13 to determine the position of the optical fiber guidewire in the body cavity and to accurately determine the direction of subsequent movement of the optical fiber guidewire.

Referring to FIG. 15, a and b represent two gratings in opposite directions. In the practices, laser light is transmitted by the optical fiber and is scattered into the body cavity by the grating. In addition, the grating also can collect retro-reflected laser light. Laser light from the grating a is scattered by the cavity wall and then coupled into the optical fiber via the grating a, while the laser light from the grating b is scattered by the cavity wall, and then coupled into the optical fiber via grating b. In the case of a branch cavity at the grating a, the distance between the grating a and the cavity wall is greater than the distance between the grating b and the cavity wall, and thus the time for collecting the scattered pulses by grating a is lagging behind that by grating b. In the case of a branch cavity at grating b, the distance between the grating b and the cavity wall is greater than the distance between the grating a and the cavity wall, and thus the time for collecting the scattered pulse by grating b is lagging behind that by the grating a. In this way, by analyzing the waveform of the scattered echo, the branch morphology of the cavity can be obtained, thereby guiding the shaping section 3 to bend to entry into the branch cavity. By analyzing the waveform of the grating echo in different directions, the situation of the branch cavity where each grating is located can be determined, thereby providing more detailed judgment data for the path having complicated shape of the cavity, so as to improve the efficiency of movement of the guidewire.

In the summary, the medical guidewire provided in this disclosure includes an equal-diameter fiber 2 and a variable-diameter sleeve 1 surrounding the equal-diameter fiber 2. The variable-diameter sleeve 1 includes a shaping section 3, a supporting section 5 and a pushing section 6 that are connected in sequence. The shaping section 3, the supporting section 5 and the pushing section 6 have outer diameters increased sequentially. Among them, the shaping section 3 has the smallest diameter, and thus is more bendable compared to the supporting section 5 and the pushing section 6, which prompts the shaping section 3 to guide the whole guidewire to advance along the bent blood vessel. The diameter of the supporting section 5 is larger than the diameter of the shaping section 3, which can make it have sufficient elasticity to support the movement of the shaping section 3 in blood vessels. The diameter of the pushing section 6 is larger than the diameters of the shaping section 3 and the supporting section 5, which can make it have sufficient rigidity to provide forward driving force for the shaping section 3 and the supporting section 5 in blood vessels. In addition, in order to improve the bending performance and operability of the medical guidewire, an asymmetric structure is provided on the variable-diameter sleeve 1 itself or the surround of the variable-diameter sleeve 1 along the equal-diameter fiber 2, so that the medical guidewire can be easily manipulated and enter smaller blood vessels and branch blood vessels having a large opening angle, thereby improving the effect of minimally invasive interventional treatment.

In the description, the expressions “equal”, “same” and the like are not strictly mathematical and/or geometrical limitations, and also include tolerances in manufacturing or use that can be understood by those skilled in the art.

Unless otherwise specified, the numerical range herein includes not only the entire range within its two endpoints, but also several sub-ranges contained therein.

Although preferred embodiments and examples of the present disclosure have been shown in details with reference to drawing, the present disclosure do not limited to the above embodiments and examples. Various modifications may be made without departing from the concept of the present disclosure, within the knowledge possessed by those skilled in the art.

1. A medical guidewire, comprising an equal-diameter fiber (2) and a variable-diameter sleeve (1) surrounding the equal-diameter fiber (2), wherein the variable-diameter sleeve (1) comprises a shaping section (3) capable of bending, a supporting section (5) capable of supporting an advancement of the shaping section (3), and a pushing section (6) connected to an operating handle; the shaping section (3), the supporting section (5) and the pushing section (6) are connected in sequence and have outer diameters increased sequentially; and
the medical guidewire is further provided with an asymmetric structure capable of directional bending of the medical guidewire.

2. The medical guidewire according to claim 1, wherein the equal-diameter fiber (2) is located at an axis of the variable-diameter sleeve, and the asymmetric structure is an asymmetric tube wall structure of the variable-diameter sleeve (1).

3. The medical guidewire according to claim 2, wherein the asymmetric tube wall structure is an asymmetric slit opened on the variable-diameter sleeve (1); and
the asymmetric slit is a spiral slit, and has a width that gradually decreases with spiral patterns in a direction from the shaping section (3) to the pushing section (6), or
the asymmetric slit is a rectangular slit, and has a depth on one side of the variable-diameter (1) sleeve less than a depth on other side of the variable-diameter sleeve (1).

4. The medical guidewire according to claim 2, wherein the asymmetric structure of the variable-diameter sleeve (1) lies in an inner wall of the variable-diameter sleeve (1), and an asymmetric array gap structure (9) capable of directionally bending the medical guidewire to one side is formed between the inner wall of the variable-diameter sleeve (1) and the equal-diameter fiber (2).

5. The medical guidewire according to claim 2, wherein the asymmetric tube wall structure lies in an asymmetric tube wall thickness of the variable-diameter sleeve (1), and a thickness of a tube wall on one side of the variable-diameter sleeve (1) is different from a thickness of the tube wall on other side of the variable-diameter sleeve (1).

6. The medical guidewire according to claim 2, wherein the asymmetric tube wall structure lies in a shape of the variable-diameter sleeve (1), and the variable-diameter sleeve (1) is formed by a convex side (11) and a planar side (12), or is formed by a convex side (11) and a concave side, and wherein the convex portion (11) has an arched structure.

7. The medical guidewire according to claim 1, wherein the equal-diameter fiber is fixed on an inner wall on one side of the variable-diameter sleeve (1).

8. The medical guidewire according to claim 1, wherein the medical guidewire further comprises a transition section (4) having a gradually changing diameter, located between the shaping section (3) and the supporting section (5), and the diameter of the transition section (4) gradually increases along a direction from the shaping section (3) to the supporting section (5).

9. The medical guidewire according to claim 1, wherein a proximal end of the medical guidewire is an operating handle, and the operating handle is provided with a tensing device for applying pulling force on the equal-diameter fiber;
a distal end of the medical guidewire has a hemispherical structure (7); and
the proximal end of the medical guidewire is connected to the pushing section (6) of the variable-diameter sleeve (1), and the distal end of the medical guidewire is connected to the equal-diameter fiber (2) and the shaping section (3) of the variable-diameter sleeve (1).

10. The medical guidewire according to claim 1, wherein a polymer layer (8) is provided outside the variable-diameter sleeve (1), and is a hydrophilic coating or a hydrophobic coating.
